(19) United States
(12) Patent Application Publication
(10) Pub. No.: US 2024/0349716 A1
(43) Pub. Date: Oct. 24, 2024

(54) Cell Transport and Storage Composition and Method

(71) Applicant: LIFE SCIENCE GROUP LTD, Bedfordshire (GB)

(72) Inventors: Emma Buick, Bedfordshire (GB); Jenny Murray, Bedfordshire (GB); Sebastien Farnaud, Bedfordshire (GB); Derek Renshaw, Bedfordshire (GB)

(21) Appl. No.: 18/683,760

(22) PCT Filed: Aug. 15, 2022
(86) PCT No.: PCT/GB2022/052124

§ 371 Date: Feb. 14, 2024

(30) Foreign Application Priority Data
Aug. 18, 2021 (GB) 2111803.9

(51) Int. Cl. A01N 1/02

(52) U.S. Cl. CPC A01N 1/0226

(57) ABSTRACT

The invention provides a cell transport and storage composition comprising a block copolymer of polyoxypropylene and polyoxyethylene, and at least one compound selected from the group comprising carnitine and glycerol, a method of transporting or storing cells in the composition, and the use of a block copolymer of polyoxypropylene and polyoxyethylene for cell transport and/or storage at a temperature above 8° C., and a method of transporting and storing cells in a composition comprising a block copolymer of polyoxypropylene and polyoxyethylene at a temperature of more than 8° C.

### TECHNICAL FIELD OF THE INVENTION

The present invention relates to a cell transport and storage composition. In particular, the invention relates to a cell transport and storage composition comprising a block copolymer of polyoxypropylene and polyoxyethylene. The invention further relates to methods of transporting or storing cells at ambient temperature.

### BACKGROUND TO THE INVENTION

Cell culture is a critical platform for numerous biomedical research and industrial processes, including drug discovery and screening, toxicology, antibody and recombinant protein production and most recently, cell-based therapies. Despite advances in cell culture techniques and applications thereof, including elucidating pathogenic mechanisms, pharmaceutical development, toxicological evaluation and the development of cell-based therapies, the standard method for transporting and storing cell cultures remains largely unchanged.

There are two conventional methods for transporting and storing cells. The first conventional method is to cryopreserve cells by controlled rate cooling of the cells to at least −80° C. and then using solid carbon dioxide (i.e. ‘dry ice’) or liquid nitrogen for transport. Frozen cells can also be transported using a dry shipping device (i.e. a dry vapour shipper). The second conventional method is to transport cells as growing cultures in a sealed cell culture vessel. These methods present logistical and biological challenges for the sender and the receiver.

Cryopreservation involves submerging the cells in a cryopreservation medium comprising a cryoprotective agent (CPA) prior to cooling the cells to cryogenic temperatures using controlled rate freezing. CPAs are used to eliminate, or significantly reduce, ice crystal formation when cooling cells to cryogenic temperatures. There are significant disadvantages associated with cryopreservation.

A commonly used CPA is dimethyl sulphoxide (DMSO). The process of cryopreservation for transport or storage must be carefully controlled and requires the cells to be slowly frozen at approximately 1° C. per minute to −80° C. using a controlled rate cryo-freezing container, before being transported using dry ice or transferred to liquid nitrogen for storage. Recovery of the cells requires the cells to be thawed quickly to avoid the formation of ice crystals. The CPA must then be removed or substantially diluted. Disadvantageously, dilution of the CPA must occur slowly and carefully to avoid irreversible damage to the cells by osmotic shock. In practice, this is time-consuming and difficult to consistently achieve which, therefore, disadvantageously affects the academic and commercial viability of a number of applications of cell culture technology.

A further drawback of cryopreservation is that the process is difficult to standardise across a significant number of cell types. This is due to specific requirements of different cell types when undergoing cryopreservation, the downstream application of the cells and any cell-specific specialist equipment that is to be used in downstream applications. Significant research must, therefore, be carried out for each specific cell type to be transported and stored using cryopreservation, which is costly, time consuming and involves complex risk management and experience to carry out.

Moreover, despite its widespread use as a CPA, DMSO is cytotoxic and prolonged exposure is associated with cellular changes. For example, using DMSO as a CPA increases the expression of the pro-apoptotic genes BAX (Bcl-2 Associated X-protein) and BAD, with down regulation of the anti-apoptotic gene BLC-2 (B-cell lymphoma 2) following exposure to DMSO. Moreover, extensive changes to microRNAs and the epigenetic landscape in hepatic and cardiac cells occur even when exposed to low levels of DMSO, i.e. around 0.1% DMSO.

Further, cryopreservation induced cell death (also referred to as Cryopreservation-Induced Delayed-Onset Cell Death (CIDOCD)) and delayed re-entry into the cell cycle are common occurrences in many cell types and can be observed from a few hours to several days following recovery of the cells after cryopreservation. Cryopreservation has been shown to have varying effects on the potency of different T cell populations. This has the potential to influence patient outcomes in chimeric antigen receptor T-cell therapy (CAR-T) applications.

In addition, sub-optimal cryopreservation can lead to diminished cellular functionality and reduced cell yield following cell recovery, and also, potentially, the selection of sub-populations of cells with genetic or epigenetic characteristics divergent from the original cell line. Sub-optimal cryopreservation may include cryopreservation of cells where the temperature of the cell sample has not been maintained at the correct temperature for transportation, i.e. −80° C.

The disadvantages associated with cryopreservation results in a significant reduction in the mean cell viability and cell count of cells following recovery of a cryopreserved cell sample. In some instances, the mean viability of cryopreserved cells following a recovery period of 24 hours can be as low as 40%.

There are also significant logistical challenges in respect of transporting dry ice, liquid nitrogen and dry vapour shippers.

As an alternative to transporting cryopreserved cells, the cells may be transported as growing cultures in sealed cell culture vessels. Transporting growing cultures in sealed cell culture vessels is only recommended for periods of up to 24 hours to maintain satisfactory cell count, a high level of cell viability and membrane integrity. Cells stored or transported in sealed cell culture vessels for longer than 24 hours often exhibit a reduced cell count following recovery, low cell viability and diminished cell membrane integrity. This can present significant drawbacks with respect to logistics and scheduling of transportation.

Moreover, when transported using sealed cell culture vessels, cells often become detached from an extracellular matrix (ECM), and any neighbouring cells, within the cell culture vessel. This can lead to anoikis, which is the induction of apoptosis in cells upon loss of attachment to the ECM and neighbouring cells. Additional drawbacks include potential loss of vessel integrity through physical stress, or the vessel not being thoroughly sealed. In both instances, there is a significant risk of contamination and/or leaking of the cells out of the vessel during transportation and storage.

Further, many known cell storage and transport media are xenogeneic, i.e. the media comprises material derived from a different species to that of its intended use, therefore, the media is not genetically or immunologically compatible. This significantly limits the potential applications of the cell storage and transport media.

As such, there is a need to develop an alternative method of transporting and storing cells which does not rely on cryopreservation techniques or transporting growing cultures in sealed cell culture vessels.

Embodiments of the present invention seek to ameliorate these or other disadvantages and/or to provide an improved composition and method for transporting and storing cells.

It is an aim of embodiments of the invention to overcome or mitigate at least one problem of the prior art, whether expressly disclosed herein or not.

### SUMMARY OF THE INVENTION

According to a first aspect of the invention, there is provided a cell transport and storage composition comprising a block copolymer of polyoxypropylene and polyoxyethylene, and at least one compound selected from the group comprising carnitine and glycerol.

The term ‘cell’ as used herein refers to one or more cell(s), a population of cells, or a tissue.

The term ‘storage’ as used herein in the context of cells, refers to a period of time between formulation of the cell(s) in a composition according to the invention, and either a further processing step or the clinical use of the cell(s). The term ‘storage’ may, preferably, mean short-term storage of cells. However, the composition according to the invention may also be used for long-term storage of cells. Short-term storage is typically the storage of cells for up to 120 hours. Long-term storage is typically the storage of cells for up to 12 months but can be up to several years or decades.

The term ‘formulation’ refers to a cell, a population of cells, or a tissue, with a volume of a composition according to the invention to provide a cell or tissue preparation that is suitable for clinical use, e.g., for administration to a subject.

Providing a cell transport and storage composition according to the invention advantageously provides greater, or at least sufficiently comparable, mean cell viability and cell count following cell recovery compared to that of cells transported and stored using conventional cryopreservation techniques.

In contrast to conventional solutions and cell storage/transportation techniques, it has surprisingly been found that the composition according to the invention allows cells to be transported and/or stored at ambient or room temperature (i.e. more than 8° C., for example from 8° C. to about 35° C., from about 15° C. to about 30° C., from about 20° C. to about 28° C., from about 22° C. to about 26° C., or about 25° C.). As such, the composition of the invention does not require cells to be cryogenically frozen, therefore, ameliorating many of the disadvantages of conventional cryopreservation cell transport and storage techniques.

The composition according to the invention does not require cells to be cryogenically preserved, therefore, the invention provides significant cost and logistical benefits compared with conventional cryopreservation techniques associated with cell transport and storage.

Further advantageously, cells stored and transported using the composition according to the invention recover to the pre-transport and pre-storage mean cell viability and cell count far more quickly than cells stored and transported using conventional techniques. In contrast to conventional cell storage and transport solutions and cell storage/transportation techniques, it has surprisingly been found that the composition according to the invention provides a greater, or at least sufficiently comparable, mean cell viability and cell count for periods of at least 24 hours, at least 48 hours, at least 96 hours, and even at least 120 hours, compared with conventional cell storage and transport solutions and techniques. As such, the composition of the invention provides a significant advantage over conventional transport and storage techniques, in particular transporting growing cell cultures in sealed cell culture vessels.

Beneficially, the composition according to the invention does not require the use of a cryoprotective agent. As such, the disadvantages associated with the use of a cryoprotective agent, for example the cytotoxicity of DMSO, are eliminated by providing the composition according to the invention.

The compositions according to the invention are useful for transporting cells, cell populations and tissues after their formulation to a clinical site. Advantageously, the compositions provided herein support cell survival and significantly reduce metabolic and shear stress during transport. As such, the composition according to the invention provides significant advantages over conventional methods of cell transport and storage, including growing cultures in sealed cell culture vessels.

The use of ambient temperature transit avoids the need for expensive transport procedures, cryo-preservation procedures and CPAs, avoiding subsequent CIDOCD and ensuring rapid cellular recovery post-transportation. Advantageously, the composition according to the invention overcomes economic and logistical challenges currently prohibitive to both research and medical sciences.

The compositions according to the invention are widely compatible with various cell types, cell populations, and tissues, including, but not limited to, CHO cells, HEK cells, HepG2 cells, Jurkat cells, K562 cells, HeLa cells and A549 cells. It is also envisaged that the compositions according to the invention are compatible with many additional human and animal cell lines, including primary cells, induced pluripotent stem cells (iPSCs) and stem cells. In some embodiments, the composition does not include at least one, and preferably all, of an antibiotic, fetal bovine serum and MEM (Minimum essential medium).

Carnitine may be present in the form of either of its two enantiomers, D-carnitine (S-(+)-) and L-carnitine (R-(−)-).

Carnitine may be recombinant carnitine.

Carnitine may be present in an amount of between about 0.01 and about 0.1 mM, between about 0.02 and about 0.08 mM, between about 0.03 and about 0.07 mM, between about 0.04 and about 0.06 mM, or about 0.05 mM, per litre of the cell transport and storage composition.

Carnitine may be present in an amount of at least 0.01 mM, 0.02 mM, 0.03 mM, 0.04 mM, 0.05 mM, 0.06 mM, 0.07 mM, 0.08 mM, 0.09 mM, or at least 0.1 mM, per litre of the cell transport and storage composition.

Carnitine may be present in an amount of no more than 0.01 mM, 0.02 mM, 0.03 mM, 0.04 mM, 0.05 mM, 0.06 mM, 0.07 mM, 0.08 mM, 0.09 mM, or no more than 0.10 mM, per litre of the cell transport and storage composition.

Glycerol may be present in an amount of between about 0.01 and about 0.20 mM, between about 0.02 and about 0.19 mM, between about 0.03 and about 0.18 mM, between about 0.04 and about 0.17 mM, between about 0.05 and about 0.16 mM, between about 0.06 and about 0.15 mM, between about 0.07 and about 0.14 mM, between about 0.08 and about 0.13 mM, between about 0.09 and about 0.12 mM, between about 0.10 and about 0.12 mM, or about 0.11 mM, per litre of the cell transport and storage composition.

Glycerol may be present in an amount of at least 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM, or at least 0.20 mM, per litre of the cell transport and storage composition.

Glycerol may be present in an amount of no more than 0.01 mM, 0.02 mM, 0.03 mM, 0.04 mM, 0.05 mM, 0.06 mM, 0.07 mM, 0.08 mM, 0.09 mM, 0.10 mM, 0.11 mM, 0.12 mM, 0.13 mM, 0.14 mM, 0.15 mM, 0.16 mM, 0.17 mM, 0.18 mM, 0.19 mM, or no more than 0.20 mM, per litre of the cell transport and storage composition.

Beneficially, the amounts of carnitine and glycerol which may be used in the composition according to the invention are comparably low compared to ingredients used in conventional cell transport and storage composition. As such, the present invention provides a cost-effective alternative to conventional cell transport and storage compositions.

Further beneficially, carnitine modulates the pathway for the turnover of membrane phospholipid fatty acids in intact human red blood cells. Moreover, carnitine also improves the membrane stability of red blood cells when such cells are subjected to high shear stress, such as that often experienced during transportation. Further, carnitine supports oxidative metabolism and free fatty acid utilisation intracellularly.

Advantageously, glycerol stabilises cell membranes and scavenges free radicals. Further advantageously, glycerol acts as an osmotic buffering agent.

As such, through biophysical and metabolic actions, carnitine and glycerol each support cell survival and significantly reduce metabolic and shear stress during cell transport.

The cell transport and storage composition according to the invention may be xeno-free, i.e. the composition may be non-xenogeneic and animal serum-free, so it does not contain material from any species other than human origin. Advantageously, this means that the composition according to the invention has a broader range of applications than that of known cell storage and transport media which are not xeno-free. In certain embodiments of the invention comprising carnitine, the carnitine is preferably recombinant carnitine.

The block copolymer of polyoxypropylene and polyoxyethylene may be a diblock copolymer, triblock copolymer, tetrablock copolymer or a multiblock copolymer. Preferably, the block copolymer of polyoxypropylene and polyoxyethylene is a triblock copolymer. For example, the diblock copolymer of polyoxypropylene and polyoxyethylene may comprise a sequence of repeat units E (polyoxyethylene) and P (polyoxypropylene) of (E)ₐ(P)_{b}, the triblock copolymer of polyoxypropylene and polyoxyethylene may comprise a sequence of repeat units E and P of (E)ₐ(P)_{b}(E)ₐ or, (P)_{b}(E)ₐ(P)_{b}, and the tetrablock copolymer of polyoxypropylene and polyoxyethylene may comprise a sequence of repeat units E and P of (E)ₐ(P)_{b}(E)ₐ(P)_{b} or (P)_{b}(E)ₐ(P)_{b}(E)ₐ, wherein E and P represent polyoxyethylene and polyoxypropylene, respectively, and a and b represent the number of repeat units of polyoxyethylene and polyoxypropylene, respectively. In embodiments comprising a triblock or tetrablock copolymer, the number of repeat units of each polyoxyethylene portion, and of each polyoxypropylene portion, may be the same or different.

The block copolymer may comprise any number of repeat units of polyoxypropylene and polyoxyethylene.

The triblock copolymer of polyoxypropylene and polyoxyethylene may have the formula:

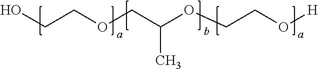

wherein a is an integer having a value of from about 1 to about 200, from about 1 to about 180, from about 1 to about 160, from about 1 to about 140, from about 1 to about 130, from about 2 to about 130, from about 10 to about 125, from about 20 to about 120, from about 30 to about 115, from about 40 to about 110, from about 50 to about 105, from about 55 to about 100, from about 60 to about 95, from about 65 to about 95, from about 70 to about 90, from about 70 to about 85, from about 70 to about 80, or about 75, or about 80, and b is an integer having a value of from about 1 to about 150, from about 3 to about 130, from about 5 to about 110, from about 7 to about 90, from about 9 to about 85, from about 11 to about 80, from about 12 to about 75, from about 13 to about 70, from about 14 to about 70, from about 15 to about 67, from about 16 to about 60, from about 17 to about 50, from about 18 to about 45, from about 19 to about 45, from about 20 to about 40, from about 22 to about 38, from about 24 to about 36, from about 26 to about 34, from about 28 to about 32, from about 29 to about 31, or about 30, or b may be an integer having a value of about 27.

The number of repeat units of each polyoxyethylene portion may be the same or different.

The ratio of percentage content in the block copolymer of polyoxyethylene to polyoxypropylene may be from about 20:1 to about 1:1, from about 18:1 to about 1:1, from about 16:1 to about 1:1, from about 14:1 to about 1:1, from about 12:1 to about 1:1, from about 10:1 to about 1:1, from about 8:1 to about 2:1, from about 7:1 to about 2:1, from about 6:1 to about 2:1, from about 5:1 to about 3:1, or, preferably, about 4:1.

The block copolymer of polyoxypropylene and polyoxyethylene may be present in an amount of between about 0.01 and about 5.0% v/v, between about 0.02 and about 4.0% v/v, between about 0.03 and about 3.0% v/v, between about 0.04 and about 2.0% v/v, between about 0.05 and about 1.0% v/v, between about 0.10 and about 0.8% v/v, between about 0.12 and about 0.6% v/v, between about 0.14 and about 0.4% v/v, between about 0.16 and about 0.35% v/v, between about 0.17 and about 0.30% v/v, between about 0.18 and about 0.25% v/v, between about 0.19 and about 0.22% v/v, or about 0.20% v/v, based on the total volume of the cell transport and storage composition.

The block copolymer of polyoxypropylene and polyoxyethylene may be present in an amount of at least about 0.05% v/v, about 0.10% v/v, about 0.15% v/v, about 0.20% v/v, about 0.25% v/v, about 0.30% v/v, about 0.35% v/v, about 0.40% v/v, about 0.45% v/v, about 0.50% v/v, about 0.55% v/v, about 0.60% v/v, about 0.65% v/v, about 0.70% v/v, about 0.75% v/v, about 0.80% v/v, about 0.85% v/v, about 0.90% v/v, about 0.95% v/v, about 1.0% v/v, about 2.0% v/v, about 3.0% v/v, about 4.0% v/v or at least about 5.0% v/v, based on the total volume of the cell transport and storage composition.

The block copolymer of polyoxypropylene and polyoxyethylene may be present in an amount of no more than about 0.05% v/v, about 0.10% v/v, about 0.15% v/v, about 0.20% v/v, about 0.25% v/v, about 0.30% v/v, about 0.35% v/v, about 0.40% v/v, about 0.45% v/v, about 0.50% v/v, about 0.55% v/v, about 0.60% v/v, about 0.65% v/v, about 0.70% v/v, about 0.75% v/v, about 0.80% v/v, about 0.85% v/v, about 0.90% v/v, about 0.95% v/v, about 1.0% v/v, about 2.0% v/v, about 3.0% v/v, about 4.0% v/v or no more than about 5.0% v/v, based on the total volume of the cell transport and storage composition.

Advantageously, at each of the concentrations of the block copolymer of polyoxypropylene and polyoxyethylene provided herein, the block copolymer stabilises the cell membrane and, therefore, protects cells against shear stress commonly experienced during transportation at ambient temperature.

The cell transport and storage composition may further comprise at least one saccharide. The at least one saccharide may be at least one monosaccharide and/or at least one disaccharide. The at least one monosaccharide may be selected from the group containing glyceraldehyde, erythrose, ribose, deoxyribose, arabinose, xylose, lyxose, glucose, D (+)-glucose monohydrate, fructose, galactose, allose, altrose, mannose, gulose, iodose, talose and sedoheptulose or mixtures thereof. The at least one disaccharide may be sucrose. Advantageously, it is believed that addition of at least one saccharide provides a source of energy to the composition according to the invention by being the primary source of intermediate metabolites for the production of energy in the citric acid cycle. Further advantageously, as the composition according to the invention allows cells to be transported and/or stored at ambient temperature, and cell metabolism is reduced at ambient temperature, the amount of saccharide, for example glucose, used in the composition is low. The reduces the cost and number of ingredients of the composition according to the invention.

The at least one monosaccharide may be present in an amount of between about 0.2 and about 100 mM, between about 0.4 and about 80 mM, between about 0.6 and about 60 mM, between about 0.8 and about 40 mM, between about 1.0 and about 20 mM, between about 2.0 and about 16 mM, between about 3.0 and about 14 mM, between about 4.0 and about 12 mM, or between about 5.0 and about 10 mM, per litre of the cell transport and storage composition. The at least one monosaccharide may be present in an amount of about 1 mM, about 2 mM, about 3 mM, about 4 mM, about 5 mM, about 6 mM, about 7 mM, about 8 mM, about 9 mM, about 10 mM, about 12 mM, about 14 mM, about 16 mM, about 18 mM or about 20 mM, per litre of the cell transport and storage composition.

The cell transport and storage composition may comprise at least one source of cations and/or at least one source of anions.

The at least one source of cations may be at least one monovalent cation. The at least one monovalent cation may be one, two, three, four, five or more than five monovalent cation(s). Advantageously, it is believed that addition of at least one monovalent cation limits unfavourable cell aggregation and maintains cell growth and phenotypic stability. Further advantageously, it is believed that the at least one monovalent cation contributes to the buffering capacity of the cell transport and storage composition. The at least one monovalent cation may be any pharmaceutically acceptable monovalent cation.

The at least one monovalent cation may comprise at least one sodium source or at least one potassium source, or a mixture thereof. The at least one sodium source may be sodium chloride. Advantageously, it is believed that sodium and/or potassium ions maintain the sodium/potassium ionic balance. It is believed that chloride ions provide a cation-anion balance within the composition according to the invention. Further advantageously, it is believed that sodium ions (for example, in the form of sodium chloride) and/or potassium ions act as an osmotically active agent maintaining the composition according to the invention at a physiological osmotic pressure and contributing to fluid osmolarity. A physiological osmotic pressure refers to an osmotic pressure that is not cytotoxic and resembles the osmotic pressure of the cell that the solution is administered to or that a cell formulated in the composition encounters in its natural environment. For most cells, a physiological osmotic pressure is about 260-345 mOsm/l, for example, 280-330 mOsm/l, 290-325 mOsm/l, 300-315 mOsm/l. In some embodiments, a physiological osmotic pressure is about 260, about 265, about 270, about 275, about 280, about 285, about 290, about 295, about 300, about 305, about 310, about 315, about 320, or about 325 mOsm/l. The at least one potassium source may be potassium chloride.

The at least one monovalent cation may be present in an amount of between about 0.5 to about 200 mM, between about 1.0 to about 190 mM, between about 1.5 to about 180 mM, between about 2.0 and about 170 mM, between about 2.5 and about 160 mM, between about 3.0 and about 150 mM, between about 3.5 and about 140 mM, between about 4.0 and about 130 mM, between about 4.5 and about 120 mM, or between about 5.0 and about 110 mM, per litre of the cell transport and storage composition.

The at least one sodium source of monovalent cations may be present in an amount of between about 10 and about 210 mM, between about 20 and about 200 mM, between about 30 and about 190 mM, between about 40 and about 180 mM, between about 50 and about 170 mM, between about 60 and about 160 mM, between about 70 and about 150 mM, between about 80 and about 140 mM, between about 90 and about 130 mM, between about 100 and about 120, or about 110 mM, per litre of the cell transport and storage composition.

The at least one potassium source of monovalent cations may be present in an amount between about 0.5 and about 20 mM, between about 1.0 and about 18 mM, between about 1.5 and about 16 mM, between about 2.0 and about 14 mM, between about 2.5 and about 12 mM, between about 3.0 and about 10 mM, between about 3.5 and about 8.0 mM, between about 4.0 and about 7.0 mM, between about 4.5 and about 6.0 mM, or about 5.0 mM, per litre of the cell transport and storage composition.

The at least one source of cations may be at least one divalent cation. The at least one divalent cation may be one, two, three, four, five or more than five sources of divalent cation. Advantageously, it is believed that addition of at least one divalent cation limits unfavourable cell aggregation and maintains cell growth and phenotypic stability. Further advantageously, it is believed that addition of at least one divalent cation contributes to electrolyte balance within the composition according to the invention which, in turn, maintains a suitable electrolytic conductivity and, therefore, maintains the ionised status of the cell membrane and the activities of enzymatic and receptor moieties. The at least one divalent cation may be any pharmaceutically acceptable source of divalent cations.

The at least one divalent cation may comprise at least one calcium source, at least one magnesium source, or a mixture thereof. The at least one calcium source and/or at least one magnesium source advantageously maintain the Ca²⁺:Mg⁺ ratio for optimal biochemical mechanisms and membrane functions. The at least one calcium source may be any pharmaceutically acceptable calcium salt selected from the group of calcium chloride, calcium chloride dihydrate, calcium hydroxide, calcium carbonate or calcium carbide, or a calcium salt formed with an acid selected from the group comprising 1-hydroxy-2-naphthoic acid, acid, 2,2-dichloroacetic 2-hydroxyethanesulphonic acid, 2-oxoglutaric acid, 4-acetamidobenzoic acid, 4-aminosalicylic acid, acetic acid, adipic acid, ascorbic acid (L), aspartic acid (L), benzenesulphonic acid, benzoic acid, camphoric acid (+), camphor-10-sulfonic acid (+), capric acid, caproic acid, caprylic acid, carbonic acid, cinnamic acid, citric acid, cyclamic acid, dodecylsulfuric acid, ethane-1,2-disulphonic acid, ethanesulphonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulphonic acid, naphthalene-1,5-disulphonic acid, naphthalene-2-sulphonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulphuric acid, tartaric acid (+L), thiocyanic acid, toluenesulphonic acid (p), and undecylenic acid, or a mixture thereof.

The at least one magnesium source may be any pharmaceutically acceptable magnesium salt selected from the group of magnesium chloride, magnesium chloride hexahydrate, magnesium hydroxide or magnesium carbonate, or a magnesium salt formed with an acid selected from the group comprising 1-hydroxy-2-naphthoic acid, 2,2-dichloroacetic acid, 2-hydroxyethanesulphonic acid, 2-oxoglutaric acid, 4-acetamidobenzoic acid, 4-aminosalicylic acid, acetic acid, adipic acid, ascorbic acid (L), aspartic acid (L), benzenesulphonic acid, benzoic acid, camphoric acid (+), camphor-10-sulfonic acid (+), capric acid, caproic acid, caprylic acid, carbonic acid, cinnamic acid, citric acid, cyclamic acid, dodecylsulfuric acid, ethane-1,2-disulphonic acid, ethanesulphonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulphonic acid, naphthalene-1,5-disulphonic acid, naphthalene-2-sulphonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulphuric acid, tartaric acid (+L), thiocyanic acid, toluenesulphonic acid (p), and undecy lenic acid, or a mixture thereof.

The at least one divalent cation may be present in an amount of between about 0.05 and about 10 mM, between about 0.10 and about 8.0 mM, between about 0.15 and about 7.0 mM, between about 0.20 and about 6.0 mM, between about 0.25 and about 5.0 mM, between about 0.30 and about 4.0 mM, between about 0.35 and about 3.0 mM, between about 0.40 and about 2.5 mM, or between about 0.45 and about 1.25 mM, per litre of the cell transport and storage composition.

The at least one calcium source may be present in an amount of between about 0.1 to about 2.4 mM, between about 0.2 and about 2.3 mM, between about 0.3 and about 2.2 mM, between about 0.4 and about 2.1 mM, between about 0.5 and about 2.0 mM, between about 0.6 and about 1.9 mM, between about 0.7 and about 1.8 mM, between about 0.8 and about 1.7 mM, between about 0.9 and about 1.6 mM, between about 1.0 and about 1.5 mM, between about 1.1 and about 1.4 mM, between about 1.2 and about 1.3 mM, or about 1.25 mM, per litre of the cell transport and storage composition.

The at least one magnesium source may be present in an amount of between about 0.1 and about 2.5 mM, between about 0.15 and about 2.0 mM, between about 0.20 and about 1.5 mM, between about 0.25 and about 1.25 mM, between about 0.30 and about 1.0 mM, between about 0.35 and about 0.75 mM, between about 0.40 and about 0.50 mM, or about 0.45 mM, per litre of the cell transport and storage composition.

The at least one source of anions may be at least one source of chloride ion or at least one source of bicarbonate ion. Advantageously, it is believed that addition of at least one source of chloride ion provides a cation-anion balance within the composition according to the invention. Further advantageously, it is believed that at least one source of bicarbonate ion acts as a pH buffering agent within the composition according to the invention. The source of the at least one chloride ion may be choline chloride, sodium chloride, potassium chloride, calcium chloride dihydrate, magnesium chloride hexahydrate, or sodium hydrogen carbonate.

The at least one source of anions may be present in an amount of between about 1 and about 250 mM, between about 5 and about 225 mM, between about 10 and about 200 mM, between about 15 and about 175 mM, between about 20 and about 150 mM, or between about 25 and about 119 mM, per litre of the cell transport and storage composition.

The at least one source of chloride ion may be present in an amount of between about 20 and about 220 mM, between about 40 and about 200 mM, between about 60 and about 180 mM, between about 80 and about 160 mM, between about 100 and about 140 mM, between about 110 and about 130 mM, between about 115 and about 125 mM, or about 119 mM, per litre of the cell transport and storage composition.

The at least one source of bicarbonate ion may be present in an amount of between about 1 and about 80 mM, between about 10 and about 60 mM, between about 15 and about 50 mM, between about 18 and about 40 mM, between about 20 and about 30 mM, between about 22 and about 27 mM, or about 25 mM, per litre of the cell transport and storage composition.

The cell transport and storage composition may further comprise an osmotically active agent. Advantageously, an osmotically active agent maintains the composition according to the invention at a physiological osmotic pressure. Advantageously, it is believed that an osmotically active agent maintains the composition according to the invention at a physiological osmotic pressure. A physiological osmotic pressure refers to an osmotic pressure that is not cytotoxic and resembles the osmotic pressure of the cell that the solution is administered to or that a cell formulated in the composition encounters in its natural environment. For most cells, a physiological osmotic pressure is from about 260 to about 345 mOsm/l, from about 280 to about 330 mOsm/l, from about 290 to about 325 mOsm/l, or from about 300 to about 315 mOsm/l. In some embodiments, a physiological osmotic pressure is about 260, about 265, about 270, about 275, about 280, about 285, about 290, about 295, about 300, about 305, about 310, about 315, about 320, or about 325 mOsm/l.

The osmotically active agent may be present in an amount of between about 50 and about 200 mM, between about 60 and about 180 mM, between about 70 and about 160 mM, between about 80 and about 140 mM, between about 90 and about 130 mM, between about 100 and about 120 mM, or about 110 mM, per litre of the cell transport and storage composition.

The osmotically active agent may be glycerol or a salt, preferably a sodium salt, a potassium salt, a calcium salt or a magnesium salt, or a source of chloride. The sodium salt may be in the form of sodium chloride.

It is believed that the osmotically active agent maintains the electrolyte balance within the composition according to the invention, which in turn maintains the osmotic gradient.

The cell transport and storage composition may further comprise at least one organic acid. It is believed that addition of at least one organic acid prevents the composition of the invention from becoming cytotoxic by maintaining the composition at a physiological pH. A physiological pH refers to a pH that is not cytotoxic and resembles the pH of the cell that the composition is administered to or that a cell formulated in the solution encounters in its natural environment. For most cells, a physiological pH is a pH of about 6.0 to about 8.0, for example a pH of 6.8-7.8, a pH of 7.0-7.7, a pH of 7.2-7.6, a pH of 7.2-7.5, or a pH of 7.4-7.5. Accordingly, in some embodiments, the composition of the invention exhibits a pH of about 7.0, about 7.1, about 7.2, about 7.3, about 7.4, about 7.5, about 7.6, about 7.7, about 7.8, about 7.9 or about 8.0.

The organic acid may be any sulphonic acid having the formula:

R—S(═O)₂—OH
wherein R is an organic alkyl or aryl group.

The sulphonic acid may be N,N-Bis (2-hydroxyethyl)-2-aminoethanesulfonic acid (BES) or N-(2-Hydroxyethyl) piperazine-N′-(2-ethanesulfonic acid) (HEPES) (4-(2-Hydroxyethyl)piperazine-1-ethanesulfonic acid).

The organic acid may be any linear or branched organic acid. The linear or branched organic acid compound may be one or more C1 to C20, or C3 to C18 linear or branched organic acid(s), or mixtures thereof. In some embodiments the linear or branched organic acid compound may be selected from the group comprising methanoic acid, acetic acid, propionic acid, acrylic acid, propiolic acid, lactic acid, 3-hydroxipropionic acid, glyceric acid, pyruvic acid, butyric acid, isobutyric acid, crotonic acid, methacrylic acid, tetrolic acid, valeric acid, isovaleric acid, pivalic acid, caproic acid, adipic acid, citric acid, sorbic acid, enanthic acid, caprylic acid, phthalic acid, pelargonic acid, trimesic acid, cinnamic acid, capric acid, hendecanoic acid, lauric acid, tridecylic acid, myristic acid, pentadecylic acid, palmitic acid, margaric acid, stearic acid, oleic acid, linoleic acid, nonadecylic acid, or arachidic acid, any of their salts or esters thereof; or any mixtures thereof.

The organic acid may be a ring-containing organic acid. The ring-containing organic acid compound may be one or more C3 to C9, or C6 to C9 ring-containing organic acid(s), or mixtures thereof. The ring-containing organic acid may comprise a ring structure selected from the group comprising a cyclic compound, a polycyclic compound, a heterocyclic compound a bicyclic compound and a spiro compound. The ring-containing organic acid may comprise at least one optionally-substituted ring structure selected from the group comprising a cyclobutene, a cyclopentane, a cyclohexane, a benzene, an ethylene oxide, an ethylenimine, a trimethylene oxide, a furan, a tetrahydrofuran, a thiophene, a pyrrole, a pyrrolidine, a pyran, a pyridine, a piperidine, a imidazole, a thiazole, a dioxane, a morpholine and a pyrimidine, or combinations thereof.

In some embodiments, the ring-containing organic acid compound may comprise a benzoic acid structure or a furanone structure.

In some embodiments the ring-containing organic acid compound may be selected from the group comprising acetyl salycilic acid (2-acetyloxybenzoic acid), salycilic acid and ascorbic acid ((2R)-2-[(1S)-1,2-dihydroxyethyl]-3,4-dihydroxy-2H-furan-5-one), or any of their salts or esters thereof; or any mixtures thereof.

The at least one organic acid may be present in an amount of between about 0.1 and about 50 mM, between about 0.5 and about 45 mM, between about 1.0 and about 40 mM, between about 1.5 and about 35 mM, between about 2.0 and about 30 mM, between about 2.5 and about 25 mM, between about 3.0 and about 20 mM, between about 3.5 and about 15 mM, between about 4.0 and about 10 mM, between about 4.5 and about 7.5 mM, between about 4.5 and about 6.0 mM, or about 5.0 mM, per litre of the cell transport and storage composition.

The at least one organic acid may be present in an amount of at least about 1.0 mM, about 2.0 mM, about 3.0 mM, about 4.0 mM, about 5.0 mM, about 6.0 mM, about 7.0 mM, about 8.0 mM, about 9.0 mM or about 10 mM, per litre of the cell transport and storage composition.

Beneficially, organic acids are relatively cheap to produce. As such, manufacture of the inventive composition is relatively cheap in comparison to compounds which rely on complex synthetic chemicals that are often expensive to produce, or cell transport and storage methods which rely on the use of dry ice or liquid nitrogen dry shippers, which are expensive to manufacture and pose significant logistical challenges.

The cell transport and storage composition may further comprise at least one amino acid or ionic form thereof or derivative thereof. Advantageously, amino acids support cell metabolism, provide cellular energy and nutrients, and may be important intermediaries in various biological pathways involving nitrogenous metabolism.

The at least one amino acid may be selected from the group containing glutamine, glutamic acid and aspartic acid, or mixtures thereof, or ionic forms thereof. For example, the composition according to the invention may comprise the ionic form of glutamic acid, i.e. glutamate, and/or the ionic form of aspartic acid, i.e. aspartate, which are a key compounds in cellular metabolism. Moreover, in certain embodiments of the invention comprising glutamic acid, or its ionic form, it is preferable that glutamic acid is in the isomeric form of L-glutamic acid. In certain embodiments of the invention comprising aspartic acid, it is preferable that aspartic acid is in the isomeric form of L-aspartic acid. However, either isomeric form, L or D, of glutamic acid, aspartic acid or glutamine may be used. Further advantageously, aspartate is a precursor to several amino acids, including methionine, threonine, isoleucine, and lysine, which are essential for the functioning of cells.

The at least one amino acid may be present in an amount of at least 0.005 mM, 0.01 mM, 0.015 mM, 0.02 mM, 0.025 mM, 0.03 mM, 0.035 mM, 0.04, 0.045 mM, 0.05 mM, 0.1 mM, 0.2 mM, 1.0 mM, 2.0 mM, 10 mM, 20 mM, 40 mM, 60 mM, 80 mM, 100 mM, 150 mM, 200 mM, 250 mM, 300 mM, 350 mM, 400 mM, 450 mM, or at least 500 mM, per litre of the cell transport and storage composition.

In certain embodiments comprising aspartic acid, or its ionic form, aspartate, the aspartic acid, or aspartate, may be present in an amount of between about 0.001 and about 0.02 mM, between about 0.002 and about 0.018 mM, between about 0.003 and about 0.017 mM, between about 0.004 and about 0.016 mM, between about 0.005 and about 0.015 mM, between about 0.006 and about 0.014 mM, between about 0.007 and about 0.013 mM, between about 0.008 and about 0.012 mM, between about 0.009 and about 0.011 mM, or about 0.01 mM, per litre of the cell transport and storage composition.

In certain embodiments comprising aspartic acid, or its ionic form, aspartate, the aspartic acid, or aspartate, may be present in an amount of at least 0.001 mM, 0.002 mM, 0.003 mM, 0.004 mM, 0.005 mM, 0.006 mM, 0.007 mM, 0.008 mM, 0.009 mM, or at least 0.01 mM, per litre of the cell transport and storage composition.

In certain embodiments comprising glutamine, the glutamine may be present in an amount of between about 10 and about 500 mM, between about 50 and about 450 mM, between about 75 and about 400 mM, between about 100 and about 375 mM, between about 120 and about 360 mM, between about 140 and about 350 mM, between about 160 and about 340 mM, between about 170 and about 330 mM, between about 180 and about 320 mM, between about 190 and about 310 mM, or between about 200 and about 300 mM, per litre of the cell transport and storage composition.

In certain embodiments comprising glutamine, the glutamine may be present in an amount of at least 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or at least 300 mM, per litre of the cell transport and storage composition.

In certain embodiment comprising glutamic acid, or its ionic form, glutamate, the glutamic acid, or its ionic form, glutamate, may be present in an amount of between about 10 and about 600 mM, between about 50 and about 550 mM, between about 75 and about 500 mM, between about 100 and about 475 mM, between about 120 and about 460 mM, between about 140 and about 450 mM, between about 160 and about 440 mM, between about 170 and about 430 mM, between about 180 and about 420 mM, between about 190 and about 410 mM, between about 200 and about 400 mM, between about 210 and about 390 mM, between about 220 and about 380 mM, between about 230 and about 370 mM, between about 240 and about 360 mM, between about 250 and about 350 mM, between about 260 and about 340 mM, between about 270 and about 330 mM, between about 280 and about 320 mM, between about 290 and about 310 mM, or about 300 mM, per litre of the cell transport and storage composition.

In certain embodiment comprising glutamic acid, or its ionic form, glutamate, the glutamic acid, or its ionic form, glutamate, may be present in an amount of at least 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, or at least 300 mM, per litre of the cell transport and storage composition.

The cell transport and storage composition may further comprise glutamic acid, or its ionic form, glutamate, and glutamine in a ratio of from about 10:2 to about 1:1, from about 9:2 to about 1:1, from about 8:2 to about 1:1, from about 7:2 to about 1:1, from about 6:2 to about 1:1, from about 5:2 to about1: 1, from about 4:2 to about 1:1, or about 3:2.

The cell transport and storage composition may further comprise a source of choline or salt thereof. The source of choline may be choline chloride or a phospholipid that incorporates choline as a headgroup, for example phosphatidyl choline. Choline is the common name of 2-trimethyl amino 1-ethanol, a quaternary amine, and is accompanied by a counterion. Choline is an important compound involved with membrane chemistry (e.g., phosphatidyl choline) and intercellular (neurotransmitter acetyl choline) communication. It is believed that the relatively large effective (hydrated) ionic size and low diffusion rate through the cell membrane of choline are important characteristics. As such, other quaternary amines or molecules positively charged at physiological pH may also be useful compounds in the composition according to the invention.

In certain embodiments comprising a source of choline or salt thereof, the choline or salt thereof may be present in an amount of between about 0.001, and about 0.02 mM, between about 0.002 and about 0.018 mM, between about 0.004 and about 0.016 mM, between about 0.006 and about 0.014 mM, between about 0.008 and about 0.012 mM, between about 0.009 and about 0.011 mM, or about 0.01 mM.

In certain embodiments comprising a source of choline or salt thereof, the choline or salt thereof may be present in an amount of at least 0.001 mM, 0.002 mM, 0.003 mM, 0.004 mM, 0.005 mM, 0.006 mM, 0.007 mM, 0.008 mM, 0.009 mM, or at least 0.01 mM, per litre of the cell transport and storage composition.

The cell transport and storage composition may further comprise thiamine or a derivative thereof. Thiamine is also known as vitamin B1. It is believed that thiamine, or a derivative thereof, acts as a coenzyme in the catabolismof sugars and amino acids and, therefore, helps maintain cell viability and function during transport and storage. Moreover, it is also believed that thiamine, or a derivative thereof, prevents pyruvic aldehyde accumulation and cell toxicity.

The thiamine or a derivative thereof may be selected from thiamine monophosphate (ThMP), thiamine pyrophosphate (TPP), thiamine triphosphate (ThTP), adenosine thiamine triphosphate (AThTP), and adenosine thiamine diphosphate (AThDP). Advantageously, TPP acts in vivo as the coenzyme of enzymes executing several vital metabolic processes such as pentose phosphate pathway and the TCA cycle. Beneficially, thiamine or a derivative thereof, in particular TPP, helps maintain cell viability and function during transport and storage.

The thiamine or derivative thereof may be present in an amount of between about 5 and about 100 nM, between about 10 and about 80 nM, between about 15 and about 70 nM, between about 20 and about 60 nM, between about 25 and about 55 nM, between about 30 and about 50 nM, between about 35 and about 45 nM, or about 40 nM, per litre of the cell transport and storage composition.

The thiamine or derivative thereof may be present in an amount of at least about 5 nM, about 10 nM, about 15 nM, about 20 nM, about 25 nM, about 30 nM, about 35 nM, about 40 nM, about 45 nM, or at least about 50 nM, per litre of the cell transport and storage composition.

The cell transport and storage composition may further comprise insulin. The insulin may be recombinant human insulin. Advantageously, insulin, in particular recombinant human insulin, facilitates glucose uptake for cell metabolism and, therefore, helps to maintain cell viability.

The recombinant human insulin may be present in an amount of between about 2 and about 50 mIU, between about 4 and about 48 mIU, between about 6 and about 46 mIU, between about 8 and about 44 mIU, between about 10 and about 42 mIU, between about 12 and about 40 mIU, between about 14 and about 38 mIU, between about 16 and about 36 mIU, between about 18 and about 34 mIU, between about 20 and about 34 mIU, between about 22 and about 34 mIU, between about 24 and about 32 mIU, between about 26 and about 30 mIU, or about 28 mIU, per litre of the cell transport and storage composition.

In embodiments, where cells formulated in a composition according to the invention are transported or stored under refrigerated conditions, e.g., at a temperature below ambient temperature (for example between 2° C. and 8° C.), the use of mobile refrigeration equipment is preferred for transport and storage. Such equipment includes, but is not limited to, insulated transport or shipment containers, wet ice packs, cooling gels, cooling containers, and mobile refrigeration units.

In addition to cell transport and storage, the composition according to the invention may also be used for irrigation of surgical fields including wound cleansing, debris removal from surgical fields and post-surgery adhesion prevention, cell reconstruction, for example of cryopreserved or pelleted cells, or administration of cells to a subject, for example cell implantation or transplantation.

According to a second aspect of the invention, there is provided a use of a block copolymer of polyoxypropylene and polyoxyethylene for cell transport and/or storage at a temperature above 8° C.

The temperature above 8° C. may be a temperature between about 8° C. and about 35° C., between about 10° C. and about 30° C., between about 15° C. and about 28° C., between about 20° C. and about 28° C., between about 22° C. and about 26° C., or about 25° C.

The temperature above 8° C. may be a temperature of about 9° C., about 10° C., about 11° C., about 12° C., about 13° C., about 14° C., about 15° C., about 16° C., about 17° C., about 18° C., about 19° C., about 20° C., about 22° C., about 24° C., about 26° C., about 28° C., about 30° C., about 32° C., or about 35° C.

The temperature above 8° C. may be a temperature of at least about 9° C., about 10° C., about 11° C., about 12° C., about 13° C., about 14° C., about 15° C., about 16° C., about 17° C., about 18° C., about 19° C., about 20° C., about 22° C., about 24° C., about 26° C., about 28° C., about 30° C., about 32° C., or at least about 35° C.

The temperature above 8° C. may be a temperature of no more than about 9° C., about 10° C., about 11° C., about 12° C., about 13° C., about 14° C., about 15° C., about 16° C., about 17° C., about 18° C., about 19° C., about 20° C., about 22° C., about 24° C., about 26° C., about 28° C., about 30° C., about 32° C., or no more than about 35° C.

The invention according to the second aspect may optionally include any of the optional features of the invention according to the first aspect. The invention according to the second aspect may also be associated with any of the advantages of any of the features, including optional features, of the invention according to the first aspect.

According to a third aspect of the invention, there is provided a method of transporting or storing cells, the method comprising transporting or storing cells in a composition comprising a block copolymer of polyoxypropylene and polyoxyethylene at a temperature of more than 8° C.

The method of the third aspect, in some embodiments, may comprise, in any order, contacting a cell, a population of cells, or a tissue, with a volume of a cell storage and transport composition to obtain a cell or tissue preparation that is suitable for clinical use, e.g., for administration to a subject, and storing the cell, population of cells or tissue for a period of up to at least 12 hours, 24 hours, 48 hours, 72 hours, 96 hours, or up to at least 120 hours, at ambient temperature, for example at a temperature of more than 8° C.

Storing the cell, population of cells or tissue may be for a period of up to at least about 1 hour, about 2 hours, about 3 hours, about 4 hours, about 5 hours, about 6 hours, about 8 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or up to at least about 120 hours.

Storing the cell, population of cells or tissue may be for a period of up to about 1 hour, about 2 hours, about 3 hours, about 4 hours, about 5 hours, about 6 hours, about 8 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or up to about 120 hours.

Storing the cell, population of cells or tissue may be for a period of up to no more than about 1 hour, about 2 hours, about 3 hours, about 4 hours, about 5 hours, about 6 hours, about 8 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, about 48 hours, about 50 hours, about 52 hours, about 54 hours, about 56 hours, about 58 hours, about 60 hours, about 62 hours, about 64 hours, about 66 hours, about 68 hours, about 70 hours, about 72 hours, about 74 hours, about 76 hours, about 78 hours, about 80 hours, about 82 hours, about 84 hours, about 86 hours, about 88 hours, about 90 hours, about 92 hours, about 94 hours, about 96 hours, about 98 hours, about 100 hours, about 102 hours, about 104 hours, about 106 hours, about 108 hours, about 110 hours, about 112 hours, about 114 hours, about 116 hours, about 118 hours, or up to no more than about 120 hours.

Storing the cell, population of cells or tissue may be for a period of more than 120 hours, for example at least 150 hours, 180 hours, 210 hours, 240 hours, 270 hours, 300 hours, 330 hours, or at least 360 hours.

The temperature of more than 8° C. may be a temperature between about 8° C. and about 35° C., between about 10° C. and about 30° C., between about 15° C. and about 28° C., between about 20° C. and about 28° C., between about 22° C. and about 26° C., or about 25° C.

The temperature of more than 8° C. may be a temperature of about 9° C., about 10° C., about 11° C., about 12° C., about 13° C., about 14° C., about 15° C., about 16° C., about 17° C., about 18° C., about 19° C., about 20° C., about 22° C., about 24° C., about 26° C., about 28° C., about 30° C., about 32° C., or about 35° C.

The temperature above 8° C. may be a temperature of at least about 9° C., about 10° C., about 11° C., about 12° C., about 13° C., about 14° C., about 15° C., about 16° C., about 17° C., about 18° C., about 19° C., about 20° C., about 22° C., about 24° C., about 26° C., about 28° C., about 30° C., about 32° C., or at least about 35° C.

The temperature of more than 8° C. may be a temperature of no more than about 9° C., about 10° C., about 11° C., about 12° C., about 13° C., about 14° C., about 15° C., about 16° C., about 17° C., about 18° C., about 19° C., about 20° C., about 22° C., about 24° C., about 26° C., about 28° C., about 30° C., about 32° C., or no more than about 35° C.

The method may comprise transporting or storing cells in a composition according to the first aspect of the invention.

The invention according to the third aspect may optionally include any of the features of the invention according to the first aspect or the second aspect.

According to a fourth aspect of the invention, there is provided a kit of parts comprising:
a. a composition comprising a block copolymer of polyoxypropylene and polyoxyethylene:
b. instructions for contacting a cell, cell population or tissue with the composition of (a.) to generate a cell preparation: and
c. a container for the contacting of (b.) and/or for storing the cell preparation of (b.).

In some embodiments, the composition of (a.) and the container of (c.) are suitable for use of the cell preparation of (b.) for transplantation to a subject.

The invention according to the fourth aspect may optionally include any of the features of the invention according to the first aspect, second aspect or third aspect.

In order that the invention may be more clearly understood one or more embodiments thereof will now be described, by way of example only, with reference to the accompanying drawings, of which:

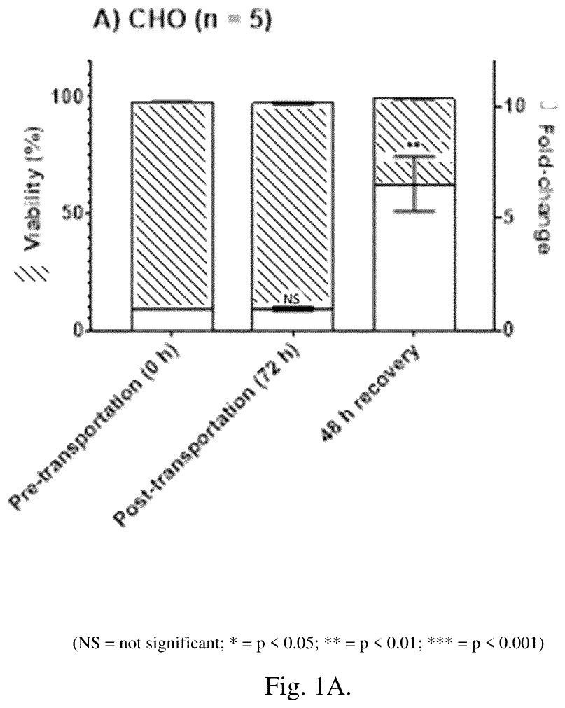

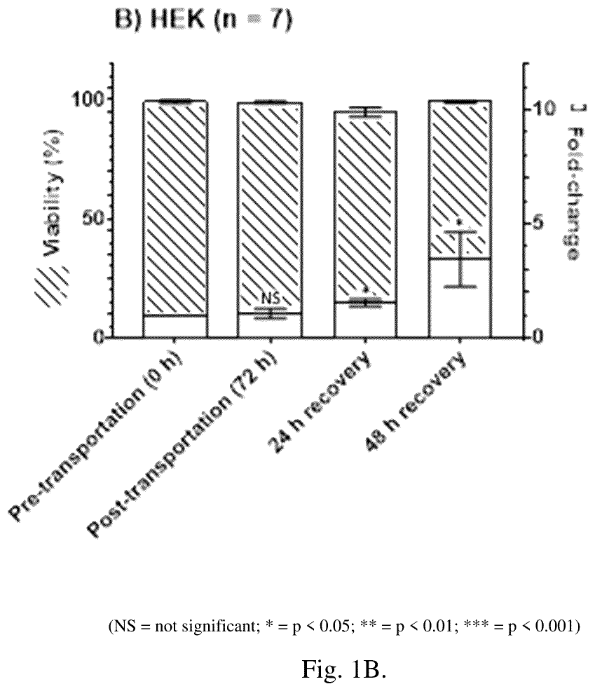

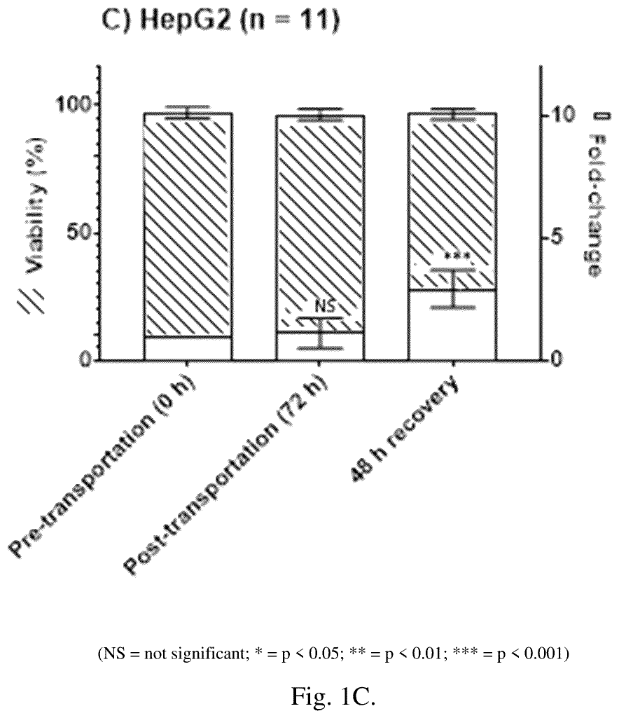

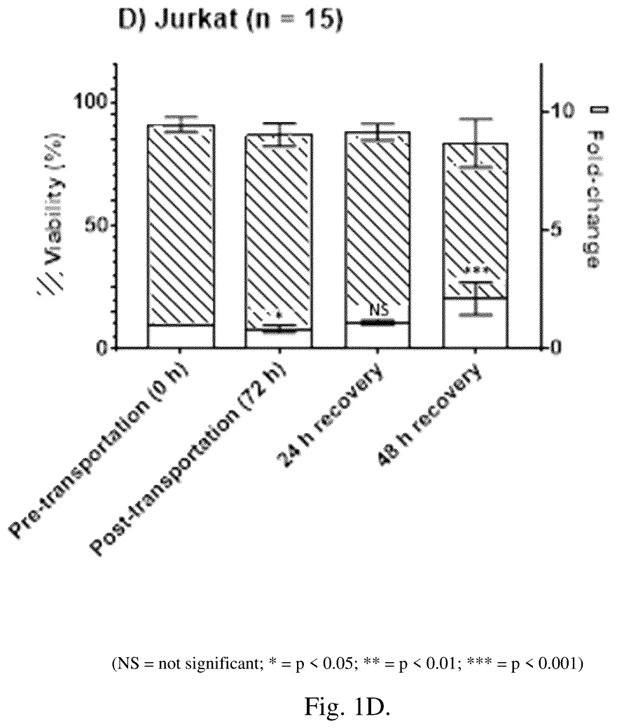

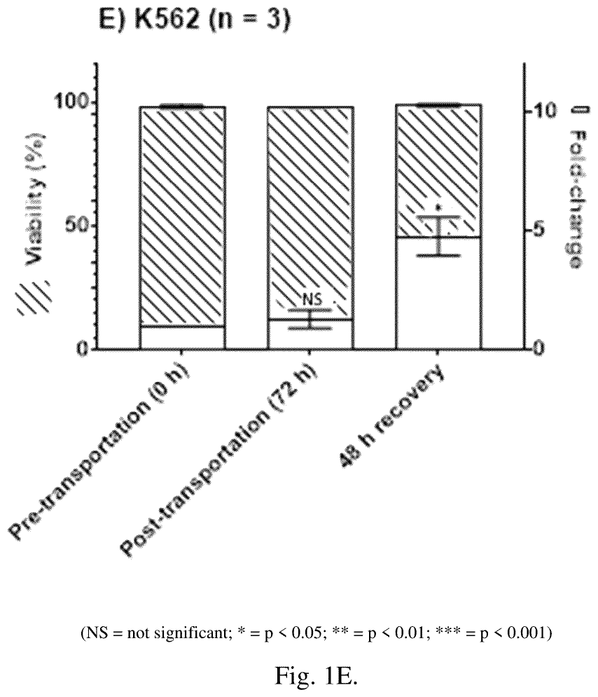

FIG. 1A is a graph showing the cell viability (%) and total cell count fold-change of CHO (n=5) cells pre-transportation, post-transportation and following a 48 hour recovery, when stored and transported in a composition according to the invention for 72 hours,

FIG. 1B is a graph showing the cell viability (%) and total cell count fold-change of HEK293 (n=7) cells pre-transportation, post-transportation and following a 24 hour recovery and a 48 hour recovery, when stored and transported in a composition according to the invention for 72 hours,

FIG. 1C is a graph showing the cell viability (%) and total cell count fold-change of HepG2 (n=11) cells pre-transportation, post-transportation and following a 48 hour recovery, when stored and transported in a composition according to the invention for 72 hours,

FIG. 1D is a graph showing the cell viability (%) and total cell count fold-change of Jurkat (n=15) cells pre-transportation, post-transportation and following a 24 hour recovery and a 48 hour recovery, when stored and transported in a composition according to the invention for 72 hours,

FIG. 1E is a graph showing the cell viability (%) and total cell count fold-change of K562 (n=3) cells pre-transportation, post-transportation and following a 48 hour recovery, when stored and transported in a composition according to the invention for 72 hours,

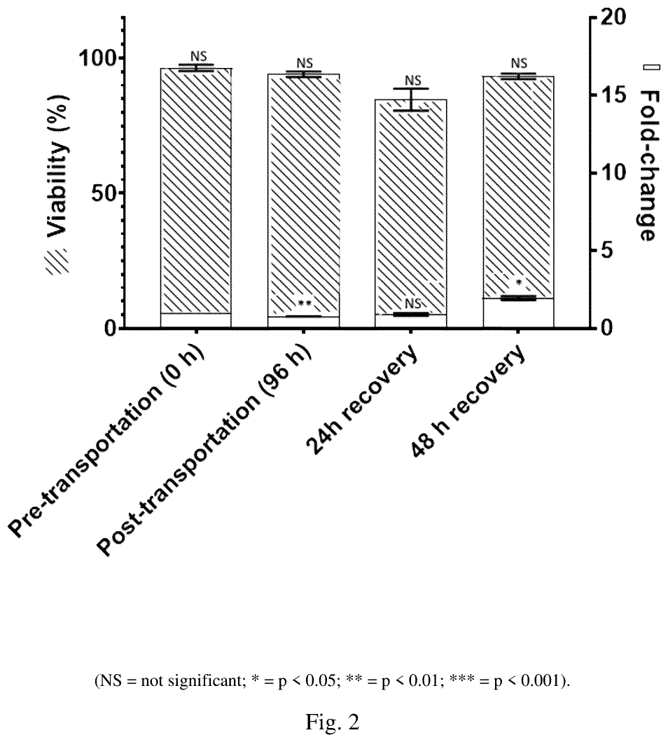

FIG. 2 is a graph showing the cell viability (%) and total cell count fold-change of Jurkat (n=3) cells pre-transportation, post-transportation and following a 24 hour recovery and a 48 hour recovery, when stored and transported in a composition according to the invention for 96 hours,

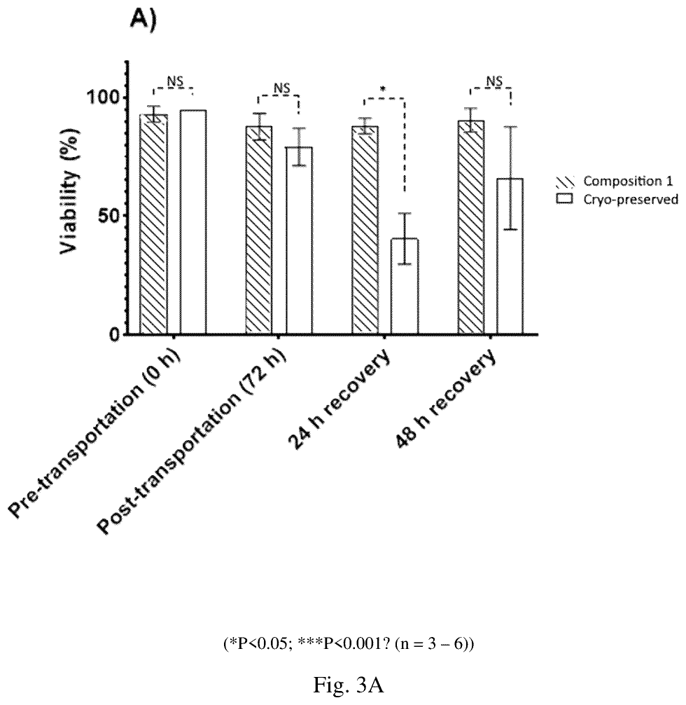

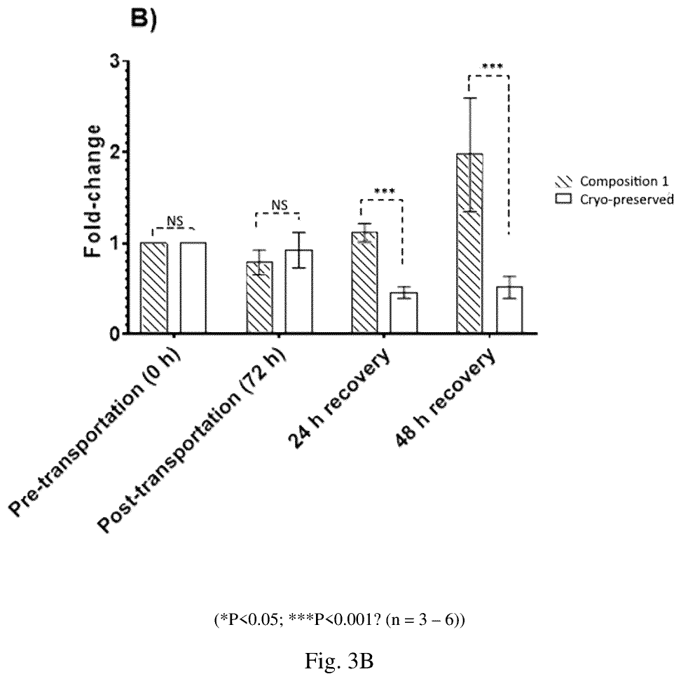

FIG. 3A is a graph showing the cell viability (%) of Jurkat cells pre-transportation, post-transportation and following a 24 hour and a 48 hour recovery, when stored and transported in a composition according to the invention, and compratively in liquid nitrogen for 72 hours,

FIG. 3B is a graph showing the total cell count fold-change in viable cell count of Jurkat cells pre-transportation, post-transportation and following a 24 hour and a 48 hour recovery, when stored and transported in a composition according to the invention and comparatively in liquid nitrogen for 72 hours,

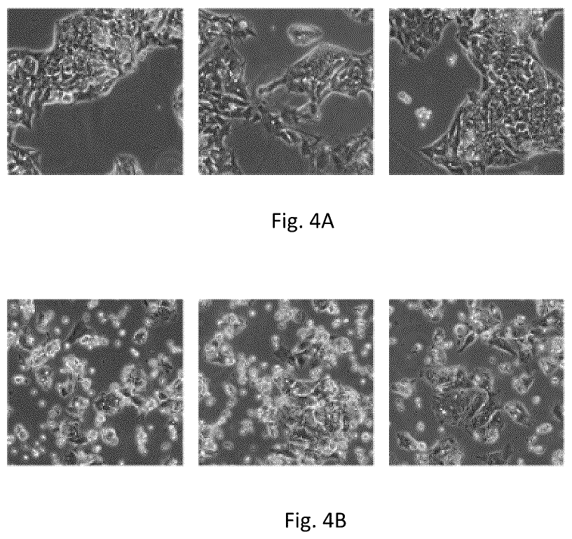

FIG. 4A shows cell morphology of HepG2 cells following storage and transportation in a composition according to the invention and recovery,

FIG. 4B shows cell morphology of HepG2 cells following storage and transportation using cryopreservation and subsequent recovery,

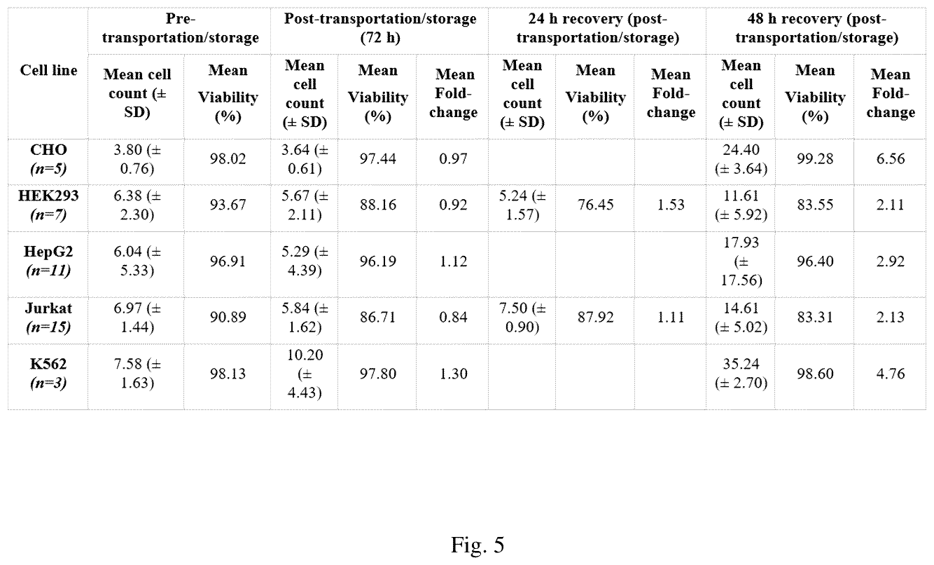

FIG. 5 is a table (Table 2) showing the mean total cell count (×10⁶ cells/mL) and viability (%) of CHO, HEK293, HepG2, Jurkat, and K562 cells pre- and post-transportation for 72 hours in a composition according to the invention and following 24 hour and 48 hour recovery,

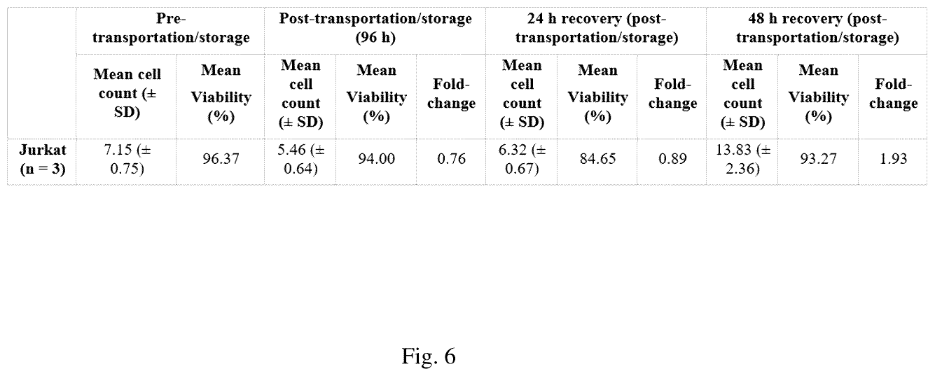

FIG. 6 is a table (Table 3) showing the mean total cell count (×10⁶ cells/mL) and viability (%) of Jurkat cells pre- and post-transportation for 96 hours in a composition according to the invention and following 24 hour and 48 hour recovery, and

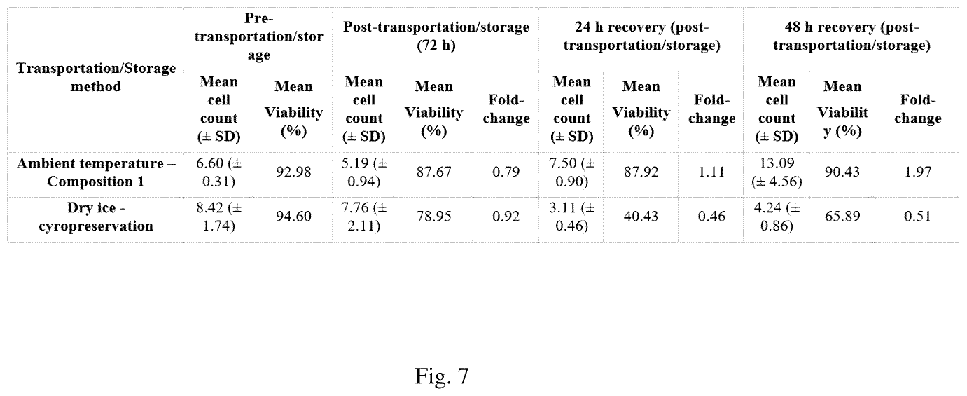

FIG. 7 is a table (Table 4) showing the mean total cell count (×10⁶ cells/mL) and viability (%) of CHO, HEK293, HepG2, Jurkat, and K562 cells pre- and post-transportation for 72 hours in a composition according to the invention at ambient temperature or in dry ice, and following 24 hour and 48 hour recovery following a return to standard culture conditions.

### EXAMPLES

Tests were carried out using five cell types; HEK293, CHO, HepG2, K562 and Jurkat cells.

The tests were carried out using a composition (Composition 1) according to the invention dissolved in water. Composition 1 comprised the following:

| | Composition 1 | |
|---|---|---|
| Sodium chloride | 110 | mM |
| Potassium ions | 5 | mM |
| Calcium ions | 1.25 | mM |
| Magnesium Ions | 0.45 | mM |
| Chloride Ions | 119 | mM |
| Bicarbonate ions | 25 | mM |
| Organic acid | 5 mM | (BES) |
| Glucose | 10 | mM |
| Glycerol | 0.11 | mM |
| Glutamate | 300 | mM |
| Glutamine | 400 | mM |
| Aspartate | 20 | μmols/L |
| Carnitine | 50 | μmols/L |
| Choline | 10 | μmols/L |
| Thiamine Pyrophosphate | 40 | nmols/L |
| Compound 1 | 0.20% | (v/v) |

Potassium ions were in the form of potassium chloride.

Calcium ions were in the form of calcium chloride dihydrate.

Magnesium ions were in the form of magnesium chloride hexahydrate.

Bicarbonate ions were in the form of sodium hydrogen carbonate.

The organic acid used was n,n-bis 2-hydroxyethyl-2-aminoethanesulfonic acid (BES).

Composition 1 comprised a triblock copolymer of polyoxypropylene and polyoxyethylene having the following formula (Compound 1):

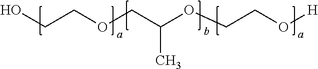

wherein

a is an integer having a value of 75, and

b is an integer having a value of 30.

### Cell Culture and Recovery Conditions

Cells were cultured at 37° C. in a humidified environment at 5% CO₂.

HEK293 and HepG2 cells were cultured in Minimum Essential Media (MEM), 1% anti-anti, 4 mM L-Glutamine, 1% non-essential amino acids (NEAA) (Gibco, Thermo Fisher Scientific, UK) and 10% foetal bovine serum (FBS; Life Science Group, UK).

CHO cells were cultured in Ham's F-12K (Kaighn's) medium, 1% anti-anti, 4 mM L-Glutamine (Gibco, Thermo Fisher Scientific, UK) and 10% FBS (Life Science Group, UK).

K562 and Jurkat cells were cultured in Roswell Park Memorial Institute (RPMI), 1% anti-anti, 4 mM L-Glutamine, sodium pyruvate (Gibco, Thermo Fisher Scientific, UK) and 10% FBS (Life Science Group, UK).

All cell lines were cultured in T25 vented culture flasks (Nunc, Thermo Fisher Scientific, UK).

### Cell Count and Viability

Cell counts and viability were assessed (minimum five measurements per sample) with 0.4% trypan blue staining (Thermo Fisher Scientific, UK) using a CytoSmart™ automated cell counter (Corning, UK) with disposable cell counting slides (Immune Systems, UK). 50 μL of the final cell suspension was taken from each sample to assess the cell count and viability.

### Transport and Storage in Composition 1

HEK293 (n=7), HepG2 (n=11), CHO (n=5), K562 (n=3), and Jurkat cells (n=14) were shipped at ambient temperature (Table 1) over a period of 72 hours or 96 hours. Samples were taken for cell count and viability (as described) pre-transport/storage and post-transport/storage.

Cultured cells were prepared immediately prior to transportation. Suspension cell lines (K562, Jurkat) were harvested directly, whilst adherent cell lines (HEK293, HepG2, CHO) were washed with Versene solution (Gibco, Thermo Fisher Scientific, UK) and dissociated using TrypLE (Gibco, Thermo Fisher Scientific, UK). Cell suspensions were pelleted at 180×g, washed with 3 mL Composition 1 and suspended in a final volume of 2 mL Composition 1. The 2 mL Composition 1 suspension was contained in a 2 mL Nalgene cryovial (Thermo Fisher Scientific, UK) and packaged securely in a polystyrene transport container. Internal package temperature during transportation was recorded using a TinyTag (Gemini Data Loggers, UK) directly adjacent to the sample vial. Transportation was performed by commercial courier service (FedEx, UK) or by car for a minimum distance of 70 mile (112 km).

TABLE 1
Temperatures experienced during transport/storage in
Composition 1. recorded using TinyTag data loggers.

| | Max temp | Min temp | Temp range |
|---|---|---|---|
| Cell type | (° C.) | (° C.) | (° C.) |
| HEK293 | 23.5 | 15 | 8.5 |
| | 26.2 | 16.7 | 9.5 |
| | 22.3 | 16.7 | 5.6 |
| | 27.4 | 13.5 | 13.9 |
| | 24.6 | 14.5 | 10.1 |
| | 22 | 11.9 | 10.1 |
| | 21.2 | 11.9 | 9.3 |
| CHO | 20.7 | 10.8 | 9.9 |
| | 23 | 11.4 | 11.6 |
| K562 | 20.5 | 6.5 | 14 |
| | 20.4 | 5.4 | 15 |
| HepG2 | 19.5 | 13.3 | 6.2 |
| | 20.4 | 12.6 | 7.8 |
| | 21.8 | 10.9 | 10.9 |
| | 22.2 | 13.5 | 8.7 |
| | 20.5 | 6.5 | 14 |
| | 20.4 | 5.4 | 15 |
| Jurkat | 23.7 | 11.8 | 11.9 |
| | 23.8 | 17.4 | 6.4 |
| | 22.8 | 17.4 | 5.4 |
| | 21.1 | 13.9 | 7.2 |
| | 27.4 | 13.5 | 13.9 |

### Transport and Storage of Cryo-Preserved Cells

Jurkat (n=6) and HepG2 (n=3) cells were harvested as previously described, pelleted at 180×g for 5 minutes and resuspended in 1 mL ice-cold FBS (Life Science Group, UK)+10% DMSO (Thermo Fisher Scientific, UK). Cell suspensions were immediately stored in 2 mL Nalgene cryovial (Thermo Fisher Scientific, UK), frozen overnight at −80° C. using a CoolCell® alcohol-free cell freezing container (BioScision, UK) and then transferred to liquid nitrogen for final cryopreservation.

For transportation, cryo-preserved cells were removed from liquid nitrogen and immediately placed in a polystyrene container containing 9 kg of dry ice. Transportation was performed by commercial courier service (FedEx, UK) or by car for a minimum distance of 70 mile (112 km). For assessing cell counts and viability, samples were taken (as described) pre-transportation and post-transportation.

### Post-Transportation Recovery and Sampling

Post-transportation Composition 1 suspensions were transferred directly into an appropriate growth media for recovery.

The growth media used for each of the cell types tested was as follows:

| Cell type | Growth media |
|---|---|
| HEK293 cells and HepG2 cells | Minimum Essential Media (MEM), 1% |
| | anti-anti, 4 mM L-glutamine, 1% non- |
| | essential amino acids (NEAA) (Gibco, |
| | Thermo Fisher Scientific, UK) and 10% |
| | foetal bovine serum (FBS; Life Science |
| | Group, UK) |
| CHO cells | Ham's F-12K (Kaighn's) medium, 1% |
| | anti-anti, 4 mM L-glutamine (Gibco, |
| | Thermo Fisher Scientific, UK) and 10% |
| | FBS (Life Science Group, UK) |
| K562 cells and Jurkat cells | Roswell Park Memorial Institute |
| | (RPMI), 1% anti-anti, 4 mM L- |
| | glutamine, sodium pyruvate (Gibco, |
| | Thermo Fisher Scientific, UK) and 10% |
| | FBS (Life Science Group, UK) |

Cryo-preserved cells were removed from dry-ice and thawed at 37° C. (less than 2 minutes). Thawed cells were suspended through the dropwise addition of 1 mL pre-warmed growth media (as above), followed by further gentle addition up to 5 mL. Cells were pelleted at 180×g for 5 minute and resuspended in pre-warmed growth media for recovery.

Post-transportation samples in Composition 1 suspensions were taken immediately before the samples were transferred into complete growth media and incubated in standard culture conditions (37° C. in a humidified environment at 5% CO₂). Assessment of cell recovery was performed at 24 hours and 48 hours.

### Morphological Analysis

Morphological analysis was performed on HepG2 cells (n=3) by phase contrast microscopy using a Cytation 5 Cell Imagining Multi-mode reader (BioTek, UK) at 48 h post-transportation recovery for both Composition 1 and cryo-preserved cells. Characteristics examined included overall cell adherence and growth morphology.

### Statistical Analysis

Fold-change in total cell count was calculated at each sample point relative to the starting total cell count (pre-transportation/storage). A dilution factor was applied to samples post-recovery to correct for initial flask inoculation volume. Fold-change and viability at respective sample-points were compared using two-way repeated measures ANOVA (or mixed model) using GraphPad Prism version 8.4.3 for Windows, GraphPad Software, San Diego, California USA.

### Results

Efficacy of Composition 1 transportation and storage over a 72 hour period was assessed using five cell lines (CHO, HEK293, HepG2, Jurkat, and K562 cells). Temperatures during transportation ranged between 5.4° C.-27.4° C., with a maximum temperature range of 15° C. in a single experiment (Table 1). Mean total cell count (×10⁶ cells/mL), viability (%) and fold-change were calculated (FIGS. 1 and 5, Table 2).

CHO cells (n=5: FIG. 1A) starting mean cell count was 3.80±0.76 (cell viability 98.02%). At initial recovery following 72 hour transport and storage, there was a non-significant mean fold change of 0.97 (cell viability 97.44%). Following 48 hour recovery culture cell count had increased with a mean fold-change of 6.56 (cell viability 99.28%).

HEK293 cells (n=7; FIG. 1B) starting mean cell count was 6.38±2.30 (cell viability 93.67%). At initial recovery following 72 hour transport and storage, there was a non-significant mean fold change of 0.92 (cell viability 88.16%). 24 hour recovery and 48 hour recovery culture had significant mean fold-changes of 1.53 (cell viability 76.45%) and 2.11 (cell viability 83.55%) respectively.

HepG2 cells (n=11: FIG. 1C) starting mean cell count was 6.04±5.33 (cell viability 96.91%). At initial recovery following 72 hour transport and storage, there was a non-significant mean fold change of 1.12 (cell viability 96.19%). Following 48 hour recovery culture cell count had increased with a mean fold-change of 2.92 (cell viability 96.40%).

Jurkat cells (n=15; FIG. 1D) starting mean cell count was 6.97±1.44 (cell viability 90.89%). A significant decrease in mean fold change of 0.84 (cell viability 86.71%) was observed at initial recovery following 72 hour transport and storage. 24 hour recovery and 48 hour recovery culture had significant mean fold-changes of 1.11 (cell viability 87.92%) and 2.13 (cell viability 83.31%) respectively.

K562 cells (n=3: FIG. 1E) starting mean cell count was 7.58±1.63 (cell viability 98.13%). At initial recovery following 72 hour transport and storage, there was a non-significant mean fold change of 1.30 (cell viability 97.80%). Following 48 hour recovery culture cell count had increased with a mean fold-change of 4.76 (cell viability 98.60%).

Surprisingly, it was found that storing and transporting each of the five cell lines tested in Composition 1 at ambient temperatures resulted in cells which maintained high cell viability post-transportation and after 24 hour and 48 hour recovery times.

Further assessment of the efficacy of Composition I was performed for transportation and storage over a 96 hour period using Jurkat cells.

Surprisingly, no significant change in cell viability was observed at any stage post-transportation with mean viability of 96.37%, 94.00%, 84.65%, and 93.27% pre-transport, post-transport (96 hour), 24 hour recovery and 48 hour recovery, respectively. A fold-change of 0.76 in cell count showed a significant decrease immediately post-transportation. No significance was observed in fold-change after 24 hour recovery, and, notably, a significant increase in fold change to 1.93 at 48 hour recovery (FIG. 6 (Table 3) and FIG. 2).

This shows that a cell storage and transport composition in the form of Composition 1 can be used to replace conventional cell storage and transport techniques, for example storing and transporting cells as growing cultures in sealed cell culture vessels or cryo-preserved cells. Advantageously, using a cell storage and transport composition in the form of Composition 1 provides favourable cell viability (at least for Jurkat cells) for longer periods of transportation and storage than is currently exhibited with conventional cell transport and storage techniques. Moreover, cells stored and transported using Composition 1 recover more quickly compared to cells stored using conventional cryopreservation techniques.

### Comparative Recovery Following Transportation of Cells in Composition 1 (at Ambient Temperature) and Cryo-Preserved Cells

The ambient temperature range was 6.7° C. to 19° C.

Mean total cell count (×10⁶ cells/mL), viability (%) and fold-change were calculated to compare transport/storage of Jurkat cells (n=3) in Composition 1 at ambient temperature and cryo-preserved samples in dry-ice. Pre- and post-transport/storage cell viability and total cell fold-change showed no significant difference between the two shipment methods.

Surprisingly, following transportation/storage for 72 hours and a 24 hour recovery period and 48 hour recovery period in standard culture conditions, viability of cells transported in Composition 1 remained stable, while viability decreased significantly for cryo-preserved cells following a recovery period of 24 hours and 48 hours to 40.43% and 65.89%, respectively. Notably, this shows a significant difference between the two transport/storage methods (FIG. 7 (Table 4), FIG. 3A).

Surprisingly, fold-change at 24 hours and 48 hours recovery for Composition 1 showed significant increases to 1.11 and 1.97, respectively. In contrast, cells transported via cryo-preservation decreased at 24 hours and 48 hours compared to pre-transportation levels to 0.46 and 0.51, respectively, therefore showing significant differences between storing and transporting cells in Composition 1 at ambient temperature and storing and transporting cryopreserved cells using dry ice (FIG. 7 (Table 4), FIG. 3B).

This shows that a cell storage and transport composition in the form of Composition 1 can be used to replace conventional cell storage and transport (in particular, short-term storage and transport) techniques which rely on cryo-preservation. Moreover, cells stored and transported using a cell storage and transport composition in the form of Composition 1 maintain favourable cell viability for significantly long periods of time, without the need to rely on conventional cryopreservation techniques.

FIG. 4 shows a comparative study between of HepG2 cell morphology following 48 hour recovery when stored using Composition 1 (FIG. 4A) and when stored using dry ice (FIG. 4B).

FIG. 4A shows representative images of cells recovered from transport/storage in Composition 1 for 72 hours and cultured using standard culture conditions for 48 hours. Notably, almost complete cell adherence had occurred by 48 hours and typical HepG2 growth morphology was well established.

In comparison, FIG. 4B shows representative images of cells recovered from cryopreservation and cultured using standard culture conditions for 48 hours. Some cell adherence was observed, although non-adherent cells are also observed, and, disadvantageously, cells had not re-established typical HepG2 growth morphology.

This shows that a cell storage and transport composition of the invention can be used to replace conventional cell storage and transport techniques which rely on cryo-preservation. As such, during recovery, cells stored and transported in compositions of the invention display advantageous cell adhesion and morphology, compared to cells stored and transported in dry ice.

The above data shows that cells transported and stored using a composition of the invention exhibit significant increases in both cell viability and cell numbers, compared to the same cell type when transported using cryo-preservation techniques.

Moreover, cells transported and stored using a composition of the invention display greater cell survivability (i.e. greater cell viability and cell count) compared to the same cell type transported using cryo-preservation techniques. Advantageously, this means that cells stored and transported using a composition of the invention are less likely to generate any cell selection bias during transportation compared to traditional cryopreservation.

The one or more embodiments are described above by way of example only. Many variations are possible without departing from the scope of protection afforded by the appended claims.

1. A method of transporting or storing cells, the method comprising transporting or storing cells in a composition comprising a block copolymer of polyoxypropylene and polyoxyethylene at a temperature of more than 8° C.

2. The method of transporting or storing cells according to claim 1, the method comprising transporting or storing cells in a composition comprising a block copolymer of polyoxypropylene and polyoxyethylene, and at least one compound selected from carnitine and glycerol.

3. The method of transporting or storing cells according to claim 2, wherein transporting or storing cells is at a temperature of more than 8° C.

4. The method of transporting or storing cells according to claim 1, wherein the temperature of more than 8° C. is a temperature between 8° C. and 35° C.

5. The method of transporting or storing cells according to claim 4, wherein the temperature of more than 8° C. is a temperature between 10° C. and 30° C.

6. The method of transporting or storing cells according to claim 5, wherein the temperature of more than 8° C. is a temperature between 15° C. and 25° C.

7. The method of transporting or storing cells according to claim 1, wherein the cells are transported or stored for a period of up to at least 12 hours, 24 hours, 48 hours, 72 hours, 96 hours, or up to at least 120 hours.

8. The method of transporting or storing cells according to claim 7, wherein the cells are transported or stored for a period of up to at least 72 hours, 96 hours, or up to at least 120 hours.

9. The method of transporting or storing cells according to claim 8, wherein the cells are transported or stored for a period of up to at least 120 hours.

10. The method of transporting or storing cells according to claim 1, wherein the method comprises a step of:
a. contacting a cell, a population of cells, or a tissue, with a volume of the composition to obtain a cell or tissue preparation that is suitable for clinical use.

11. The method of transporting or storing cells according to claim 10, wherein, after step (a), the method comprises the step of:
b. storing the cell, population of cells or tissue for a period of up to at least 12 hours, 24 hours, 48 hours, 72 hours, 96 hours, or up to at least 120 hours, at a temperature of more than 8° C.

12. The method of transporting or storing cells according to claim 11, wherein the temperature of more than 8° C. is a temperature between 8° C. and 35° C.

13. The method of transporting or storing cells according to claim 1, wherein the composition further comprises at least one compound selected from the group comprising carnitine and glycerol.

14. A block copolymer composition comprising polyoxypropylene and polyoxyethylene for cell transport and/or storage at a temperature above 8° C.

15. The block copolymer composition of claim 14, further comprising at least one compound selected from carnitine and glycerol.
